(19) United States
(12) Patent Application Publication
(10) Pub. No.: US 2012/0329988 A1
(43) Pub. Date: Dec. 27, 2012

(54) METHOD FOR THE EXTRACTION AND HYDROLYSIS OF ANY PROTEIN SUBSTANCE, NATURAL AUXINS AND POLYPHENOLS FROM SOURCES OF PLANT ORIGIN AND THEIR DERIVATIVES

(76) Inventors: Alberto Pons Raga, Betera (ES); Francisca Fuster Ramon, Betera (ES)

(21) Appl. No.: 13/504,414

(22) PCT Filed: Oct. 26, 2009
(86) PCT No.: PCT/ES2009/070460

§ 371 Date: Aug. 29, 2012

(51) Int. Cl. C07K 1/14

(52) U.S. Cl. 530/343

(57) ABSTRACT

Method for the extraction and hydrolysis of any protein substance, natural auxins and polyphenols from sources of plant origin and their derivatives, which from a any vegetable matter (roots, stems including bark, leaves, fruits, seeds and derivatives) and by an hydrolysis in acid alcohol which is added alcohols and mineral acids, the resulting mass is subjected to a thermodynamic treatment, with subsequent utilization of physical systems of separate solid/liquid plant extract is obtained a hydro alcoholic.

### BACKGROUND OF THE INVENTION

1. Field of the Invention

The object of this patent is to present a new method of extraction and hydrolysis of any protein substance (especially defensins, thionins and rip's (the latter inhibitory proteins called ribosomes), natural auxins and polyphenols from any source of plant origin (roots, stems including bark, leaves, fruits, seeds and derivatives) on an industrial scale.

With this new procedure which is based on acidic hydrolysis alcoholic get fractionate proteins resulting in an extract or substance which contains among other components a small peptides of molecular weight. Many of these peptides are substances commonly called defensins, thionins'S AND RIP (ribosome-inhibiting proteins). In turn, polyphenols and auxins are released from their associations generating organic substances free in solution.

The application of such an extensive range of peptides allows us to get this exhaust system as an adjunct in farming, can give plants an acquired immune system that is vital in the defense of plants against diseases caused by these supported, and also stabilizes the metabolic processes and adapt to stress or overexertion. Similarly applied, as an external, cosmetic and pharmaceutical uses to create defenses against infections, antioxidant systems and protective color of all external parts of mammals.

2. Description of the Related Art

Currently there are several plant extracts on the market, which include many products that are derived from a plant source. Each of these products has a process to obtain proper to it, and that fits the general principle of obtaining the highest concentration of substances in these terms.

There alcoholic extracts, products of acid or alkaline hydrolysis, saline extracts, solvent extracts, but not described in the prior art method for obtaining a plant extract obtained by any ALCOHOL ACID HYDROLYSIS whose purpose is fractionated vegetable protein peptides (especially defensins, thionins and RIP'S), and generate free molecules of the family of plant polyphenols and natural plant auxins.

While it is true that from ancient extraction systems both alcoholic and acid extraction have been used in laboratories and preparation methods of samples to be analyzed, is the first time a system is developed by blending two types of hydrolysis as a means of fractionation of proteins and peptides obtained from natural polyphenols auxins and industrial scale.

More specifically, as background and to explain the differences with other known hydrolysis procedures the following documents are cited which referred to hydrolysis and/or acid hydrolysis as a means of sample preparation, i.e., micro-scale preparations with laboratory equipment (small capacity):

Document 1 “SCHWARTZ, H et al. Analytica Chimica Acta in February 2009, Vol. 633, No. 2, pages 204-215” The study detailed here compares hydrolysis systems as a means of specimen preparation laboratory. What we have done, in addition to combining two sets of hydrolysis (alcohol and acid) is to establish the optimal conditions for acid hydrolysis alcohol that allows us to divide industrial proteins, ultimately we have developed an industrial process that did not exist as that for this purpose. Therefore, we designed a hydrolysis combining two guys in a very different scope of work presented in this article (laboratory versus industry) and also target different (analysis against peptic substances production).

Document 2 identified as “YILDIZ, L. Talanta et Al. October 2008, Vol. 77, No. 1, pages 304-313” The detailed study here again refers to hydrolysis as a means of preparing samples for analysis, as we have said is very different from establishing a hydrolysis at industrial level. We emphasize that this article mentions the difficulty of some flavonoids glycosides into aglycons through hydrolysis. That is, not all equally hydrolysis serves for a particular purpose. We also use the hydrolysis for industrial purposes, we have managed to merge two types and establish the conditions fractionate proteins.

Document 3 identified as “Tounsi, M. S. et al. Industrial Crops and Products. September 2009, Vol. 30, No. 2, pages 292-296.” This article also mentions the acid hydrolysis as a means of preparing samples for analysis. In this case would apply here as comments pertaining to the two previous documents.

### DETAILED DESCRIPTION OF THE EMBODIMENTS OF THE INVENTION

As an alternative to the procedures already being used in the industrial world, an extraction procedure and hydrolysis of any plant protein substance (especially defensins, thionins and RIP'S), natural plant auxins, plant polyphenols from plant sources and their derivatives was first developed, which is the object of the present invention.

In this procedure starting from a vegetable material (either roots, stems including bark, leaves, fruits, seeds and derivatives), it proceeds to an acidic alcoholic hydrolysis consisting of the addition of linear alcohols within the range from 1 carbon atom in the molecule up to 8 carbon atoms, i.e. from the methanol to octanol, together with mineral acids. In amounts ranging from 10% to 60% of the final mass obtained in the addition of alcohols, and 1% to 30% of the final mass obtained in the addition of any mineral acid, the addition of these ingredients, generates the optimal mix of which is necessary to extract and hydrolyzed vegetable protein any substance (especially defensins, thionins and RIP'S), natural plant auxins and plant polyphenols. Depending on the source and wealth of plant material used will result in a more or less liquid wealth of target substances.

The resulting mixture is subjected to conditions established thermodynamic temperature control from 50° C. to 150° C. and pressure in the range 0.1 to 3.1 atmospheres of pressure. The reaction is a simple process reflux with absolute control of temperature and pressure. Each fraction obtained peptide requires a temperature optimum of production. The diverse nature of the peptides (especially defensins, thionins and RIP's), polyphenols and natural auxins wanted us to perform temperature scaling for all these substances are produced.

The various peptides obtained can, if our interest for the product you want to obtain, then using physical systems solid/liquid separation any and concentration systems based on techniques of molecular exclusion, nanofiltration, or liquid chromatography, which will result in more concentrated plant extracts or fractionated according to molecular size of peptide components (especially defensins, thionins and RIP's) natural auxins and polyphenols.

The percentage of peptides (especially defensins, thionins and RIP's), natural auxins and polyphenols will depend on the plant raw material (or derivatives) of the splitting.

Having sufficiently described the nature of the present invention, as well as a way of putting it into practice, it may be added that the invention may undergo certain variations in the procedure and composition, provided that such alterations do not vary substantially the features that are claimed below.

What is claimed is:

1. Method for the extraction and hydrolysis of any protein substance, natural auxins and polyphenols from sources of plant origin and their derivatives, characterized in that starting from a plant material or any derivatives thereof are applicable to the implementation of a hydrolysis whereby alcoholic acid of proceeds to the fractionation of proteins.

2. Method for the extraction and hydrolysis of any protein substance, natural auxins and polyphenols from sources of plant origin and their derivatives according to claim 1, wherein the alcoholic acidic hydrolysis involves the addition of linear alcohols within the range that will from 1 carbon atom in the molecule up to 8 carbon atoms, ie from methanol to octanol, together with mineral acids in amounts ranging from 10% to 60% of the final mass obtained in the addition of alcohols, and a 1% to 30% of the final mass obtained in the addition of any mineral acid, the addition of these ingredients, generates the optimum mixture which is necessary to extract and hydrolyze all protein substances, natural auxins and plant polyphenols; by origin and wealth of the vegetable matter used or derivatives thereof, will result in a more or less liquid wealth in the structure of target substances.

3. Method for the extraction and hydrolysis of any protein substance, natural auxins and polyphenols from sources of plant origin and their derivatives according to claim 2, wherein the resulting mixture is subjected to a thermodynamic conditions established by the temperature control from 50° C. to 150° C. and pressure in the range 0.1 atmospheres to 3.1 atmospheres of pressure, even so, the kinetics of the reaction requires strict control of temperature and pressure-time for the diverse group of peptides plants, auxins natural plant and plant polyphenols sought and the wide range of temperatures is due to the diverse nature of plant peptides, natural plant auxins and plant polyphenols that wanted us to perform temperature scaling for products sought to be produced.

4. Method for the extraction and hydrolysis of any protein substance, natural auxins and polyphenols from sources of plant origin and their derivatives according to claim 3, characterized in that it allows further use of physical systems of solid/liquid separation any and concentration systems based on molecular exclusion techniques, nanofiltration, or liquid chromatography; that plant extracts can be concentrated and separated according to molecular size peptide components, natural auxins and polyphenols.
